(19) United States
(12) Patent Application Publication
(10) Pub. No.: US 2025/0319238 A1
(43) Pub. Date: Oct. 16, 2025

(54) COATING COMPOSITION FOR MEDICAL IMPLANTS TO PREVENT FOREIGN SUBSTANCE ADHESION

(71) Applicant: Lynk Solutec Inc., Seoul (KR)

(72) Inventors: Jungmok SEO, Seoul (KR); Yeontaek Lee, Seoul (KR)

(73) Assignee: Lynk Solutec Inc., Seoul (KR)

(21) Appl. No.: 19/093,803

(22) Filed: Mar. 28, 2025

(30) Foreign Application Priority Data
Apr. 16, 2024 (KR) 10-2024-0050549

(51) Int. Cl. A61L 33/06; A61L 29/08; A61L 33/00; A61L 33/04

(52) U.S. Cl. CPC A61L 33/068; A61L 29/085; A61L 33/0017; A61L 33/04; A61L 2420/06

(57) ABSTRACT

Provided is a coating composition for a medical implantable device to prevent foreign substance adhesion, and more specifically, the coating composition includes a stock solution and a diluted solution, and the stock solution includes an aliphatic hydrocarbon solvent; a siloxane compound; an alcohol; one or more amino silanes; and a silane-based oligomer.

### CROSS-REFERENCE TO RELATED APPLICATIONS

This application claims the priority of Korean Patent Application No. 10-2024-0050549 filed on Apr. 16, 2024, in the Korean Intellectual Property Office, the disclosure of which is incorporated herein by reference.

### BACKGROUND

### Field

The present disclosure relates to a coating composition for an implant to prevent foreign substance adhesion, and more particularly, to a coating composition including a stock solution and a diluted solution, in which the stock solution includes an aliphatic hydrocarbon solvent; a siloxane compound; an alcohol; one or more amino silanes; and a silane-based oligomer.

### Description of the Related Art

When a stenotic lesion occurs in the human lumen due to tumors or other causes, such as in the esophagus, duodenum, bile duct, ureter, urinary tract, or airway, the affected organ cannot function normally. In such cases, a stent can be inserted into the stenotic site to expand the narrowed passage. Stents are widely used to secure the patency of human lumens.

A ureteral stent, in particular, is a long, tubular device that supports the ureteral wall, preventing stenosis or obstruction and ensuring the passage of urine. By doing so, it improves urine flow within the ureter and helps alleviate related symptoms.

However, after stent insertion, bacteria or viruses can invade the implanted area, leading to infections such as cystitis or pyelonephritis. Additionally, complications such as thrombus formation may occur following stent placement.

Medical devices implanted in the human body serve various functions, such as compensating for lost bodily functions, supporting natural physiological activities, or locally delivering active substances. However, depending on the application, these devices come into contact with biological tissues for either short or long durations. Therefore, it is crucial for such devices to possess anti-fouling properties that prevent the adhesion of foreign substances like blood, mucus, bacteria, cells, calcifications, and food residues. Examples of implantable medical devices include stents, catheters, and medical tubing.

To address these challenges, the present inventors have developed a specialized coating composition that fundamentally prevents the adhesion of not only blood but also moisture, mucus, and bacteria. By applying this coating composition to implantable medical devices such as stents, catheters, and medical tubing, it was discovered that the composition effectively prevents foreign substance adhesion and contamination, leading to the completion of the present invention.

In this regard, Korean Patent No. 10-1159406 discloses a coating composition for preventing restenosis in blood vessels and a stent manufactured using the composition.

### SUMMARY

The present disclosure has been made to solve the problems of the related art, and an object of the present disclosure is to provide a coating composition for preventing foreign substance adhesion on implantable medical devices, including a stock solution and a diluted solution.

Another object of the present disclosure is to provide an implantable medical device coated with a foreign substance adhesion-preventing coating composition, in which the coating composition includes a stock solution and a diluted solution, and the stock solution includes an aliphatic hydrocarbon solvent; a siloxane compound; an alcohol; one or more amino silanes; and a silane-based oligomer.

As a technical solution for achieving the above-mentioned technical problem, one aspect of the present disclosure provides a coating composition for preventing foreign substance adhesion on implantable medical devices, including a stock solution and a diluted solution, in which the stock solution includes an aliphatic hydrocarbon solvent; a siloxane compound; an alcohol; one or more amino silanes; and a silane-based oligomer.

The diluted solution may include an organosilicon compound.

The weight mixing ratio of the stock solution and the diluted solution may be 1:45 to 55.

The implantable medical device may be a stent, a catheter, a subcutaneous implant, a chemical sensor, a lead, a pacemaker, a vascular graft, a wound dressing, an implantable pulse generator, or an implantable cardiac defibrillator.

The stent may be an esophageal stent, a vascular stent, a biliary stent, a gallbladder stent, a pancreatic stent, a uterine stent, a urethral stent, a ureteral stent, a lacrimal duct stent, an Eustachian tube stent, a fallopian tube stent, or a bronchial stent.

The catheter may be a drainage catheter, an infusion catheter, a parenteral feeding catheter, or a urological catheter.

Another aspect of the present disclosure provides An implantable medical device coated with a foreign substance adhesion-preventing coating composition

The implantable medical device may further include a silane-based lubricant coating applied over the coating composition.

The coating composition according to the present invention, when applied to implantable medical devices such as stents, catheters, and medical tubing, may effectively prevent the adhesion of foreign substances such as blood, mucus, bacteria, cells, calcifications, and food residues. Additionally, by preventing such foreign substance adhesion, it may help mitigate complications such as infections, inflammation, and thrombus formation that can occur during surgery, thereby enhancing surgical safety and precision.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a photograph showing an XPS survey spectrum of a coating composition according to Example of the present disclosure;

FIG. 2 is a graph showing light transmittance of a glass plate coated with a coating composition according to Example of the present disclosure;

FIGS. 3A and 3B are photographs showing results of a blood adhesion prevention experiment of coating compositions according to Example and Comparative Example of the present disclosure, respectively;

FIG. 4 is a photograph showing results of a marine organism adhesion prevention experiment of a coating composition according to Example of the present disclosure;

FIG. 5 is a photograph showing the results of a blood adhesion experiment on a medical tube according to a comparative example of the present invention.

FIG. 6 is a photograph showing the results of a blood adhesion experiment on a medical tube coated with the coating composition according to an embodiment of the present invention.

### DETAILED DESCRIPTION OF THE EMBODIMENT

Hereinafter, the present disclosure will be described in more detail. However, the present disclosure may be embodied in various different forms, and the present disclosure is not limited by embodiments described herein, and the present disclosure will be only defined by claims to be described below.

Terms used in the present disclosure are used only to describe specific embodiments, and are not intended to limit the present disclosure. A singular expression includes a plural expression unless otherwise defined differently in a context. Throughout the present specification, unless explicitly described to the contrary, ‘comprising’ a certain component means further comprising another component other than excluding the other component.

A first aspect of the present disclosure provides A coating composition for an implantable medical device, including a stock solution and a diluted solution, in which the stock solution includes an aliphatic hydrocarbon solvent; a siloxane compound; an alcohol; one or more amino silanes; and a silane-based oligomer.

Hereinafter, the coating composition for an implantable medical device according to the first aspect of the present disclosure will be described in detail.

In one embodiment of the present disclosure, the coating composition may include a stock solution and a diluted solution, and at this time, the stock solution may include an aliphatic hydrocarbon solvent, a siloxane compound, an alcohol, one or more amino silanes, and a silane-based oligomer.

In one embodiment of the present disclosure, the aliphatic hydrocarbon solvent may include a C₇ to C₁₂ aliphatic hydrocarbon solvent, and preferably a C₉ to C₁₂ aliphatic hydrocarbon solvent. In addition, the aliphatic hydrocarbon solvent may have a boiling range of about 140° C. to 220° C.

In one embodiment of the present disclosure, the content of the aliphatic hydrocarbon solvent may be 20 to 50 parts by weight, preferably 24 to 25 parts by weight, and more preferably 30 to 40 parts by weight, based on 100 parts by weight of the stock solution.

In one embodiment of the present disclosure, the siloxane compound may include a compound selected from the group consisting of polydimethylsiloxane, polyvinylsiloxane, polyphenylmethylsiloxane, hexamethyldisiloxane, and combinations thereof, and preferably may include a compound selected from the group consisting of methyl-terminated polydimethylsiloxane, vinyl-terminated polydimethylsiloxane, hydroxy-terminated polydimethylsiloxane, carboxy-terminated polydimethylsiloxane, and combinations thereof.

In one embodiment of the present disclosure, the content of the siloxane compound may be 30 to 60 parts by weight, preferably 35 to 55 parts by weight, and more preferably 40 to 50 parts by weight, based on 100 parts by weight of the stock solution.

In one embodiment of the present disclosure, the alcohol may be selected from the group consisting of, for example, methanol, ethanol, isopropanol, cyclohexanol, benzyl alcohol, 2-octanol, ethylene glycol, propylene glycol, glycerol, and combinations thereof.

In one embodiment of the present disclosure, the content of the alcohol may be 5 to 25 parts by weight, preferably 7 to 13 parts by weight, and more preferably 10 to 20 parts by weight, based on 100 parts by weight of the stock solution.

In one embodiment of the present disclosure, the amino silane may include amino silane including at least one selected from the group consisting of bis[(3-trimethoxysilyl)propyl]amine silane, N-beta-(aminoethyl)-gamma-aminopropyl-trimethoxy silane, N-(3-(trimethoxysilyl)propyl)-1,2-ethanediamine silane, N,N′-bis(3-(trimethoxysilyl)propyl)-1,2-ethanediamine silane, and N—N-bis(3-(trimethylsiloxy)propyl)-1,2-ethanediamine silane.

In one embodiment of the present disclosure, the content of the one or more amino silanes may be 5 to 20 parts by weight, preferably 7 to 17 parts by weight, and more preferably 10 to 15 parts by weight, based on 100 parts by weight of the stock solution.

In one embodiment of the present disclosure, the silane-based oligomer may include a compound formed from monomers selected from the group consisting of dimethyldimethoxysilane, vinylmethyldimethoxysilane, n-octylmethyldimethoxysilane, n-octadecylmethyldimethoxysilane, methyldimethoxysilane, 3-chloropropylmethyldimethoxysilane, 2-chloroethylmethyldimethoxysilane, allyldimethoxysilane, (3,3,3-trifluoropropyl)methyldimethoxysilane, n-propylmethyldimethoxysilane, chloromethylmethyldimethoxysilane, di-n-octyldimethoxysilane, vinyl(chloromethyl)dimethoxysilane, methylcyclohexyldiethoxysilane, vinylmethyldiethoxysilane, 1-(triethoxysilyl)-2-(diethoxymethylsilyl)ethane, n-octylmethyldiethoxysilane, octaethoxy-1,3,5-trisilapentane, n-octadecylmethyldiethoxysilane, methacryloxypropylmethyldiethoxysilane, 2-hydroxy-4-(3-methyldiethoxysilylpropoxy)diphenylketone, (3-glycidoxypropyl)methyldiethoxysilane, dodecylmethyldiethoxysilane, dimethyldiethoxysilane, diethyldiethoxysilane, 1,1-diethoxy-1-silacyclopent-3-ene, chloromethylmethyldiethoxysilane, bis(methyldiethoxysilylpropyl)amine, 3-aminopropylmethyldiethoxysilane, (methacryloxymethyl)methyldiethoxysilane, 1,2-bis(methyldiethoxysilyl)ethane, and diisobutyldiethoxysilane, vinyltrimethoxysilane, vinyltriethoxysilane, benzyltriethoxysilane, butenyltriethoxysilane, (triethoxysilyl)cyclohexane, O-(vinyloxybutyl)-N-triethoxysilylpropylcarbamate, 10-undecenyltrimethoxysilane, n-(3-trimethoxysilylpropyl)pyrrole, N-[5-(trimethoxysilyl)-2-aza-1-oxopentyl]caprolactam, (3,3,3-trifluoropropyl)trimethoxysilane, triethoxysilylundecanal ethylene glycol acetal, (S)—N-triethoxysilylpropyl-O-menthocarbamate, triethoxysilylpropylethylcarbamate, N-(3-triethoxysilylpropyl)-4,5-dihydroimidazole, (3-triethoxysilylpropyl)-t-butylcarbamate, styrylethyltrimethoxysilane, 2-(4-pyridylethyl)triethoxysilane, n-propyltrimethoxysilane, n-propyltriethoxysilane, (S)—N-1-phenylethylN′-triethoxysilylpropylurea, (R)—N-1-phenylethyl-N′-triethoxysilylpropylurea, N-phenylaminopropyltrimethoxysilane, N-phenylaminomethyltriethoxysilane, phenethyltrimethoxysilane, pentyltriethoxysilane, n-octyltrimethoxysilane, n-octyltriethoxysilane, 7-octenyltrimethoxysilane, S-(octanoyl)mercaptopropyltriethoxysilane, n-octadecyltrimethoxysilane, n-octadecyltriethoxysilane, methyltrimethoxysilane, methyltriethoxysilane, N-methylaminopropyltrimethoxysilane, 3-methoxypropyltrimethoxysilane, methacryloxypropyltrimethoxysilane, methacryloxypropyltriethoxysilane, methacryloxymethyltrimethoxysilane, methacryloxymethyltriethoxysilane, and O-(methacryloxyethyl)-N-(triethoxysilylpropyl)carbamate, tetramethoxysilane and/or tetraethoxysilane, octamethylcyclotetrasiloxane, and combinations thereof.

In one embodiment of the present disclosure, the content of the silane-based oligomer may be 0.1 to 1.0 parts by weight based on 100 parts by weight of the stock solution.

In one embodiment of the present disclosure, the diluted solution may be an organosilicon compound, and the organosilicon compound may include a compound selected from the group consisting of hexamethyldisiloxane, 1,1,3,3-tetramethyldisiloxane, vinyltrimethylsilane, methyltrimethylsilane, hexamethyldisilane, methylsilane, dimethylsilane, trimethylsilane, diethylsilane, propylsilane, phenylsilane, vinyltriethoxysilane, vinyltrimethoxysilane, tetramethoxysilane, tetraethoxysi lane, phenyltrimethoxysilane, methyltriethoxysilane, octamethylcyclotetrasiloxane, and combinations thereof.

In one embodiment of the present disclosure, the weight mixing ratio of the stock solution and the diluted solution may be 1:45 to 55, specifically 1:47 to 52, and more specifically 1:48 to 51. At this time, if the weight mixing ratio of the stock solution and the diluted solution is less than 1:45, the coating property of the coating composition may be deteriorated. In addition, if the weight mixing ratio of the stock solution and the diluted solution is more than 1:55, the content of the stock solution to be contained may be too small, reducing the effectiveness of preventing adhesion of foreign substances such as blood, mucus, and bacteria, and consequently, the contamination prevention effect for the implantable medical device may not be sufficiently achieved.

In one embodiment of the present disclosure, the coating composition may further include a silane-based lubricant. At this time, in one embodiment of the present disclosure, in the coating composition, the silane-based lubricant may be mixed with the stock solution and the diluted solution and coated together, or the coating composition in which the stock solution and the diluted solution are mixed may be first coated to form an intermediate coating layer, and then an oil film layer may also be formed on the coating layer of the stock solution and the diluted solution using the silane-based lubricant, and preferably, the oil film layer may be coated on the coating layer of the stock solution and the diluted solution using the silane-based lubricant.

In one embodiment of the present disclosure, the silane-based lubricant may include a lubricant selected from the group consisting of a polydimethylsiloxane-based lubricant, a trimethylsilyl-terminated polydimethylsiloxane-based lubricant, a vinyldimethylsilyl-terminated polydimethylsiloxane-based lubricant, an epoxycyclohexyldimethylsilyl-terminated polydimethylsiloxane-based lubricant, a glycidoxypropyldimethylsilyl-terminated polydimethylsiloxane-based lubricant, a p-styryldimethylsilyl-terminated polydimethylsiloxane-based lubricant, a methacryloxypropyldimethylsilyl-terminated polydimethylsiloxane-based lubricant, an acryloxydimethylsilyl-terminated polydimethylsiloxane-based lubricant, an aminopropyldimethylsilyl-terminated polydimethylsiloxane-based lubricant, a 3-isocyanatopropyldimethylsilyl-terminated polydimethylsiloxane-based lubricant, a 3-mercaptopropyldimethylsilyl-terminated polydimethylsiloxane-based lubricant, a 3-ureidopropyldimethylsilyl-terminated polydimethylsiloxane-based lubricant, an ethyldimethylsilyl-terminated polydimethylsiloxane-based lubricant, a propyldimethylsilyl-terminated polydimethylsiloxane-based lubricant, a hexyldimethylsilyl-terminated polydimethylsiloxane-based lubricant, an octyldimethylsilyl-terminated polydimethylsiloxane-based lubricant, a decyldimethylsilyl-terminated polydimethylsiloxane-based lubricant, a phenyldimethylsilyl-terminated polydimethylsiloxane-based lubricant, and combinations thereof.

In one embodiment of the present disclosure, the implantable medical device may be selected from the group consisting of a stent, a catheter, a subcutaneous implant, a chemical sensor, a lead, a pacemaker, a vascular graft, a dressing, an implantable pulse generator, and a cardiac defibrillator, and preferably, the implantable medical device may be a stent or a catheter.

In one embodiment of the present disclosure, the stent may be a medical stent used for various medical purposes, and preferably, the stent may be selected from the group consisting of an esophageal stent, a vascular stent, a biliary stent, a gallbladder stent, a pancreatic stent, a uterine stent, a urethral stent, a ureteral stent, a lacrimal stent, an Eustachian tube stent, a fallopian tube stent, and a bronchial stent.

In one embodiment of the present disclosure, the stent may be a plastic stent (tube stent) or a metal stent. The plastic stent (tube stent) may be made of materials such as polyurethane, polyethylene, polyethylene terephthalate, or polytetrafluoroethylene (PTFE), whereas the metal stent may be made of materials such as stainless steel, cobalt alloy, or nickel alloy.

In one embodiment of the present disclosure, the catheter may be selected from the group consisting of a drainage catheter, an infusion catheter, a suction catheter, a parenteral feeding catheter, and a urological catheter, and preferably, the catheter may be a drainage catheter, a suction catheter, a general-purpose infusion and drainage tube catheter, or a general-purpose tube catheter for urology.

In one embodiment of the present disclosure, the coating composition may be applied to the surface of the implantable medical device using methods such as spray coating, brush coating, or dip coating. When applied to the surface of the implantable medical device, the coating composition may effectively prevent the adhesion of foreign substances such as blood, mucus, and bacteria, thereby reducing the risk of complications such as infection, inflammation, and thrombosis that may occur during surgical procedures.

A second aspect of the present disclosure provides an implantable medical device coated with a coating composition for preventing foreign substance adhesion.

Detailed descriptions of portions overlapping with those of the first aspect of the present disclosure have been omitted, but the contents described for the first aspect of the present disclosure can be equally applied even if the description thereof has been omitted from the second aspect.

Hereinafter, an implantable medical device coated with a coating composition for preventing foreign substance adhesion according to the second aspect of the present disclosure will be described in detail.

In one embodiment of the present disclosure, the implantable medical device may be selected from the group consisting of a stent, a catheter, a subcutaneous implant, a chemical sensor, a lead, a pacemaker, a vascular graft, a dressing, an implantable pulse generator, and a cardiac defibrillator, and preferably, the implantable medical device may be a stent or a catheter.

In one embodiment of the present disclosure, the stent may be a medical stent used for various medical purposes, and preferably, the stent may be selected from the group consisting of an esophageal stent, a vascular stent, a biliary stent, a gallbladder stent, a pancreatic stent, a uterine stent, a urethral stent, a ureteral stent, a lacrimal stent, an Eustachian tube stent, a fallopian tube stent, and a bronchial stent.

In one embodiment of the present disclosure, the catheter may be selected from the group consisting of a drainage catheter, an infusion catheter, a suction catheter, a parenteral feeding catheter, and a urological catheter, and preferably, the catheter may be a drainage catheter, a suction catheter, a general-purpose infusion and drainage tube catheter, or a general-purpose tube catheter for urology.

In one embodiment of the present disclosure, the coating composition is a composition comprising a stock solution and a diluted solution, wherein the stock solution may include: an aliphatic hydrocarbon solvent; a siloxane compound; an alcohol; one or more amino silanes; and a silane-based oligomer.

In one embodiment of the present disclosure, the aliphatic hydrocarbon solvent may include a C₇ to C₁₂ aliphatic hydrocarbon solvent, and preferably a C₉ to C₁₂ aliphatic hydrocarbon solvent. In addition, the aliphatic hydrocarbon solvent may have a boiling range of about 140° C. to 220° C.

In one embodiment of the present disclosure, the content of the aliphatic hydrocarbon solvent may be 20 to 50 parts by weight, preferably 24 to 25 parts by weight, and more preferably 30 to 40 parts by weight, based on 100 parts by weight of the stock solution.

In one embodiment of the present disclosure, the siloxane compound may include a compound selected from the group consisting of polydimethylsiloxane, polyvinylsiloxane, polyphenylmethylsiloxane, hexamethyldisiloxane, and combinations thereof, and preferably may include a compound selected from the group consisting of methyl-terminated polydimethylsiloxane, vinyl-terminated polydimethylsiloxane, hydroxy-terminated polydimethylsiloxane, carboxy-terminated polydimethylsiloxane, and combinations thereof.

In one embodiment of the present disclosure, the content of the siloxane compound may be 30 to 60 parts by weight, preferably 35 to 55 parts by weight, and more preferably 40 to 50 parts by weight, based on 100 parts by weight of the stock solution.

In one embodiment of the present disclosure, the alcohol may be selected from the group consisting of, for example, methanol, ethanol, isopropanol, cyclohexanol, benzyl alcohol, 2-octanol, ethylene glycol, propylene glycol, glycerol, and combinations thereof.

In one embodiment of the present disclosure, the content of the alcohol may be 5 to 25 parts by weight, preferably 7 to 13 parts by weight, and more preferably 10 to 20 parts by weight, based on 100 parts by weight of the stock solution.

In one embodiment of the present disclosure, the amino silane may include amino silane including at least one selected from the group consisting of bis[(3-trimethoxysilyl)propyl]amine silane, N-beta-(aminoethyl)-gamma-aminopropyl-trimethoxy silane, N-(3-(trimethoxysilyl)propyl)-1,2-ethanediamine silane, N,N′-bis(3-(trimethoxysilyl)propyl)-1,2-ethanediamine silane, and N—N-bis(3-(trimethylsiloxy)propyl)-1,2-ethanediamine silane.

In one embodiment of the present disclosure, the content of the one or more amino silanes may be 5 to 20 parts by weight, preferably 7 to 17 parts by weight, and more preferably 10 to 15 parts by weight, based on 100 parts by weight of the stock solution.

In one embodiment of the present disclosure, the silane-based oligomer may include a compound formed from monomers selected from the group consisting of dimethyldimethoxysilane, vinylmethyldimethoxysilane, n-octylmethyldimethoxysilane, n-octadecylmethyldimethoxysilane, methyldimethoxysilane, 3-chloropropylmethyldimethoxysilane, 2-chloroethylmethyldimethoxysilane, allyldimethoxysilane, (3,3,3-trifluoropropyl)methyldimethoxysilane, n-propylmethyldimethoxysilane, chloromethylmethyldimethoxysilane, di-n-octyldimethoxysilane, vinyl(chloromethyl)dimethoxysilane, methylcyclohexyldiethoxysilane, vinylmethyldiethoxysilane, 1-(triethoxysilyl)-2-(diethoxymethylsilyl)ethane, n-octylmethyldiethoxysilane, octaethoxy-1,3,5-trisilapentane, n-octadecylmethyldiethoxysilane, methacryloxypropylmethyldiethoxysilane, 2-hydroxy-4-(3-methyldiethoxysilylpropoxy)diphenylketone, (3-glycidoxypropyl)methyldiethoxysilane, dodecylmethyldiethoxysilane, dimethyldiethoxysilane, diethyldiethoxysilane, 1,1-diethoxy-1-silacyclopent-3-ene, chloromethylmethyldiethoxysilane, bis(methyldiethoxysilylpropyl)amine, 3-aminopropylmethyldiethoxysilane, (methacryloxymethyl)methyldiethoxysilane, 1,2-bis(methyldiethoxysilyl)ethane, and diisobutyldiethoxysilane, vinyltrimethoxysilane, vinyltriethoxysilane, benzyltriethoxysilane, Butenyltriethoxysilane, (triethoxysilyl)cyclohexane, O-(vinyloxybutyl)-N-triethoxysilylpropylcarbamate, 10-undecenyltrimethoxysilane, n-(3-trimethoxysilylpropyl)pyrrole, N-[5-(trimethoxysilyl)-2-aza-1-oxopentyl]caprolactam, (3,3,3-trifluoropropyl)trimethoxysilane, triethoxysilylundecanal ethylene glycol acetal, (S)—N-triethoxysilylpropyl-O-menthocarbamate, triethoxysilylpropylethylcarbamate, N-(3-triethoxysilylpropyl)-4,5-dihydroimidazole, (3-triethoxysilylpropyl)-t-butylcarbamate, styrylethyltrimethoxysilane, 2-(4-pyridylethyl)triethoxysilane, n-propyltrimethoxysilane, n-propyltriethoxysilane, (S)—N-1-phenylethylN′-triethoxysilylpropylurea, (R)—N-1-phenylethyl-N′-triethoxysilylpropylurea, N-phenylaminopropyltrimethoxysilane, N-phenylaminomethyltriethoxysilane, phenethyltrimethoxysilane, pentyltriethoxysilane, n-octyltrimethoxysilane, n-octyltriethoxysilane, 7-octenyltrimethoxysilane, S-(octanoyl)mercaptopropyltriethoxysilane, n-octadecyltrimethoxysilane, n-octadecyltriethoxysilane, methyltrimethoxysilane, methyltriethoxysilane, N-methylaminopropyltrimethoxysilane, 3-methoxypropyltrimethoxysilane, methacryloxypropyltrimethoxysilane, methacryloxypropyltriethoxysilane, methacryloxymethyltrimethoxysilane, methacryloxymethyltriethoxysilane, and O-(methacryloxyethyl)-N-(triethoxysilylpropyl)carbamate, tetramethoxysilane and/or tetraethoxysilane, octamethylcyclotetrasiloxane and combinations thereof.

In one embodiment of the present disclosure, the content of the silane-based oligomer may be 0.1 to 1.0 parts by weight based on 100 parts by weight of the stock solution.

In one embodiment of the present disclosure, the diluted solution may be an organosilicon compound, and the organosilicon compound may include a compound selected from the group consisting of hexamethyldisiloxane, 1,1,3,3-tetramethyldisiloxane, vinyltrimethylsilane, methyltrimethylsilane, hexamethyldisilane, methylsilane, dimethylsilane, trimethylsilane, diethylsilane, propylsilane, phenylsilane, vinyltriethoxysilane, vinyltrimethoxysilane, tetramethoxysilane, tetraethoxysi lane, phenyltrimethoxysilane, methyltriethoxysilane, octamethylcyclotetrasiloxane, and combinations thereof.

In one embodiment of the present disclosure, the weight mixing ratio of the stock solution to the diluted solution may be 1:45 to 55, specifically 1:47 to 52, and more specifically 1:48 to 51. If the weight mixing ratio of the stock solution to the diluted solution is less than 1:45, the coating properties of the coating composition may degrade, and its transmittance may decrease. Conversely, if the weight mixing ratio exceeds 1:55, the content of the stock solution may be too low, reducing the effectiveness of preventing adhesion of foreign substances such as blood, mucus, and bacteria, and the contamination prevention effect for implantable medical devices such as stents and catheters may not be sufficiently achieved.

In one embodiment of the present disclosure, the coating composition may be applied to the surface of the implantable medical device using methods such as spray coating, brush coating, or dip coating.

In one embodiment of the present disclosure, the implantable medical device may be further coated with a silane-based lubricant over the coating composition.

In one embodiment of the present disclosure, the silane-based lubricant may include a lubricant selected from the group consisting of a polydimethylsiloxane-based lubricant, a trimethylsilyl-terminated polydimethylsiloxane-based lubricant, a vinyldimethylsilyl-terminated polydimethylsiloxane-based lubricant, an epoxycyclohexyldimethylsilyl-terminated polydimethylsiloxane-based lubricant, a glycidoxypropyldimethylsilyl-terminated polydimethylsiloxane-based lubricant, a p-styryldimethylsilyl-terminated polydimethylsiloxane-based lubricant, a methacryloxypropyldimethylsilyl-terminated polydimethylsiloxane-based lubricant, an acryloxydimethylsilyl-terminated polydimethylsiloxane-based lubricant, an aminopropyldimethylsilyl-terminated polydimethylsiloxane-based lubricant, a 3-isocyanatopropyldimethylsilyl-terminated polydimethylsiloxane-based lubricant, a 3-mercaptopropyldimethylsilyl-terminated polydimethylsiloxane-based lubricant, a 3-ureidopropyldimethylsilyl-terminated polydimethylsiloxane-based lubricant, an ethyldimethylsilyl-terminated polydimethylsiloxane-based lubricant, a propyldimethylsilyl-terminated polydimethylsiloxane-based lubricant, a hexyldimethylsilyl-terminated polydimethylsiloxane-based lubricant, an octyldimethylsilyl-terminated polydimethylsiloxane-based lubricant, a decyldimethylsilyl-terminated polydimethylsiloxane-based lubricant, a phenyldimethylsilyl-terminated polydimethylsiloxane-based lubricant, and combinations thereof.

In one embodiment of the present disclosure, the silane-based lubricant may be coated on the surface of the implantable medical device, which has been coated with the foreign substance adhesion-preventing coating composition, using methods such as spray coating, brush coating, or dip coating.

In one embodiment of the present disclosure, after the implantable medical device is coated with the foreign substance adhesion-preventing coating composition and the silane-based lubricant, it may be further cured through methods such as ultraviolet (UV) irradiation, plasma treatment, or heat treatment.

In one embodiment of the present disclosure, the implantable medical device may exhibit the effect of preventing the adhesion of foreign substances such as blood, mucus, and bacteria due to the coating of the foreign substance adhesion-preventing coating composition, thereby preventing contamination of the implantable medical device.

Hereinafter, Examples of the present disclosure will be described in detail to be easily implemented by those skilled in the art. However, the present disclosure can be implemented in various different forms and is not limited to Examples described herein.

### Example 1. Coating of Glass Plate Using Coating Composition

### 1. Preparation of Coating Composition

According to ingredients and contents shown in Table 1 below, a stock solution of a coating composition for preventing foreign substance adhesion on an implantable medical device was prepared, and the prepared stock solution was mixed with hexamethyldisiloxane as a diluted solution at a weight mixing ratio of 1:49 to prepare a coating composition.

TABLE 1

| | | Content |
|---|---|---|
| NO | Ingredient | (wt %) |
| 1 | Medium aliphatic solvent naphtha (petroleum) | 35 |
| 2 | Polydimethylsiloxane hydroxy-terminated | 43.5 |
| 3 | Isopropanol | 15 |
| 4 | N-(3-(Trimethoxysilyl) propyl)-1,2-ethanediamine | 5 |
| 5 | N,N′-bis(3-(trimethoxysilyl)propyl)-1,2-ethanediamine | 0.5 |
| 6 | N,N-Bis(3-(Trimethylsiloxy)propyl)-1,2-ethanediamine | 0.5 |
| 7 | Oligomers of (ethylenediaminepropyl) trimethoxysilane | 0.5 |

### 2. Coating of Glass Plate

A glass plate was prepared as a coating base, and the glass plate was immersed in the coating composition prepared in 1. above, taken out, and dried for 5 to 10 seconds, and then coated with the coating composition of 1. above, and coated again with polydimethyl siloxanes and silicones as a lubricant in the same manner to coat an oil film layer.

### Example 2. Coating of Medical Tube Using Coating Composition

A medical tube was immersed in the coating composition prepared in 1. of Example 1, taken out, and dried for 5 to 10 seconds, and then coated with the coating composition of 1. of Example 1, and coated again with polydimethyl siloxanes and silicones as a lubricant in the same manner to coat an oil film layer.

### Comparative Example 1. Preparation of General Glass Plate

An uncoated glass plate, which was the base used in 2. of Example 1, was prepared.

### Comparative Example 2. Preparation of General Medical Tube

An uncoated medical tube used in Example 2 was prepared.

### Experimental Example 1. Analysis of X-Ray Photoelectron Spectroscopy (XPS) of Coating Composition

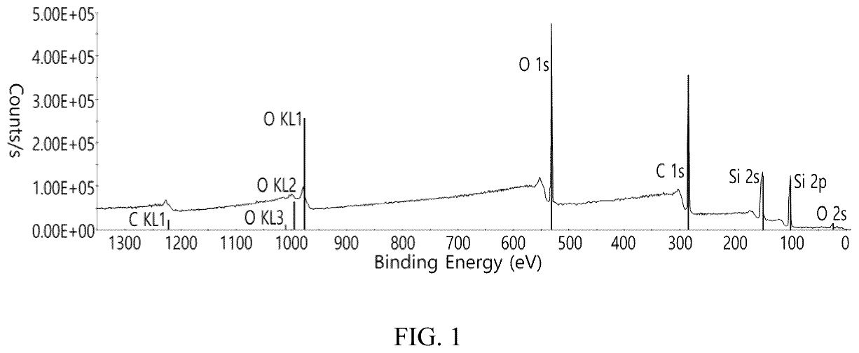

X-ray photoelectron spectroscopy (XPS) analysis was performed on the coating composition prepared in Example 1, and the results were shown in FIG. 1.

As a result of the analysis, it was confirmed that peaks appeared in C KL1, O KL3, O KL2, O KL1, O is, C is, Si 2s, Si 2p, and O 2s.

Through this, it was confirmed that the ingredients according to Table 1 of Example 1 were included.

### Experimental Example 2. Analysis of Light Transmittance of Coating Composition

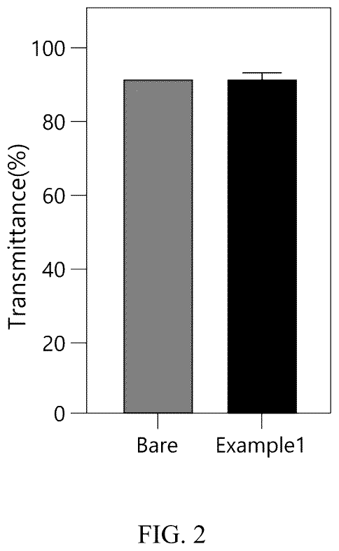

The light transmittance of the glass plate coated with the coating composition of Example 1 was measured, and the results were shown in FIG. 2. At this time, a graph shown in FIG. 2 represented the average value at UV-Vis 400 nm to 800 nm.

As shown in FIG. 2, it was confirmed that the coating composition according to Example 1 had excellent transmittance (transparency), After the implantable medical device is inserted into the body, it does not interfere with the visualization of the implant or surrounding tissues when using medical equipment such as an endoscope or ultrasound. This confirms that the coating composition is suitable for use in implantable medical devices such as stents, catheters, and other medical tubing.

### Experimental Example 3. Confirmation of Blood Adhesion Prevention Effect

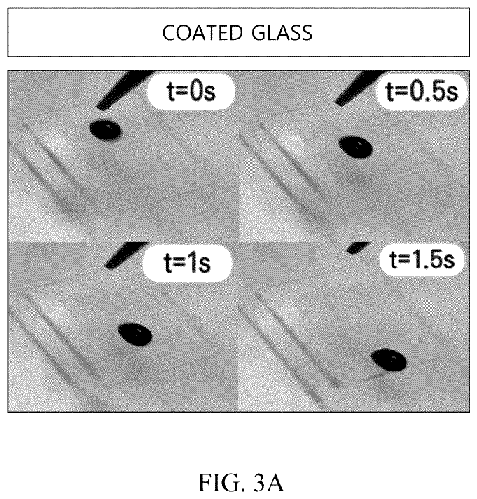

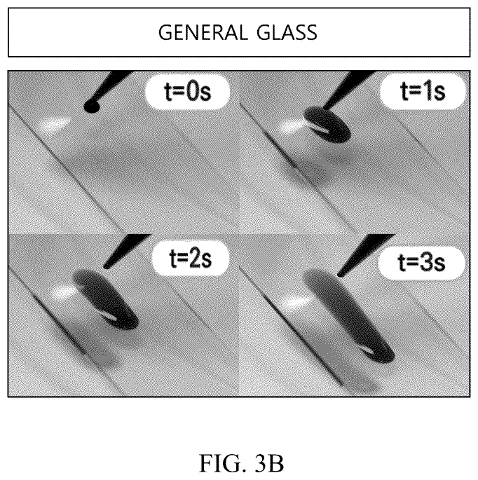

In order to confirm a blood adhesion prevention effect of the coating composition according to the present disclosure, the glass plate to which the coating layer prepared in Example 2 was applied and the uncoated glass plate of Comparative Example 1 were each inclined, and blood was dropped on the surface using a dropper, and the surface conditions of the glass plates were observed over time, which were shown in FIGS. 3A and 3B, respectively.

As shown in FIG. 3A, in the case of the glass plate coated with the coating composition of Example 2 of the present disclosure, it was confirmed that blood stains did not adhere but slid down along the slope to prevent the adhesion of biological materials. On the other hand, as shown in FIG. 3B, in the case of the glass plate of Comparative Example 1, it was confirmed that blood stains adhered.

Therefore, it was confirmed that when the coating composition of the present invention is applied to implantable medical devices such as stents, catheters, and other medical tubing, it can prevent the adhesion of foreign substances such as blood or bacteria and inhibit thrombus formation.

### Experimental Example 4. Confirmation of Maintenance Ability Under Seawater Conditions

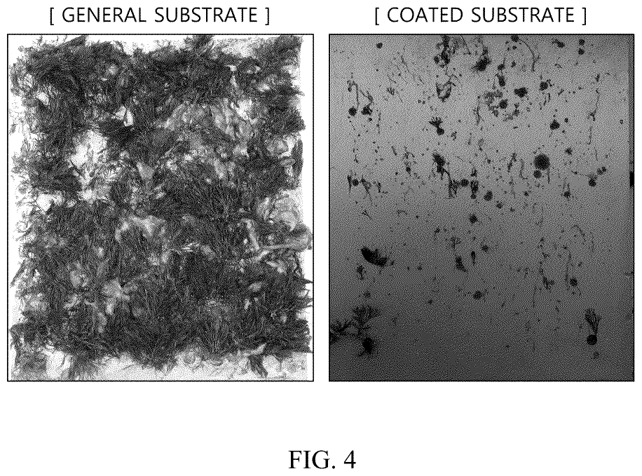

The glass plate to which the coating layer prepared in Example 1 was applied and the uncoated glass plate of Comparative Example 1 were immersed for 45 days at a depth of 3 m below the sea surface at the South Sea Research Institute of the Korea Institute of Ocean Science and Technology, and then raised above the sea surface to observe whether marine organisms adhered to the surface of the glass plate, and the results were shown in FIG. 4.

As shown in FIG. 4, it was confirmed that in the case of a substrate coated with the coating composition of the present disclosure, the adhesion degree of marine organisms was almost not shown, even though the substrate was immersed for 45 days under seawater conditions. In other words, it was confirmed that the coating composition prepared according to Example of the present disclosure was applied to have the ability to maintain the adhesion prevention effect of the marine organisms for a long time even under seawater conditions.

### Experimental Example 5. Confirmation of the Foreign Substance Adhesion Prevention Effect of the Medical Tube

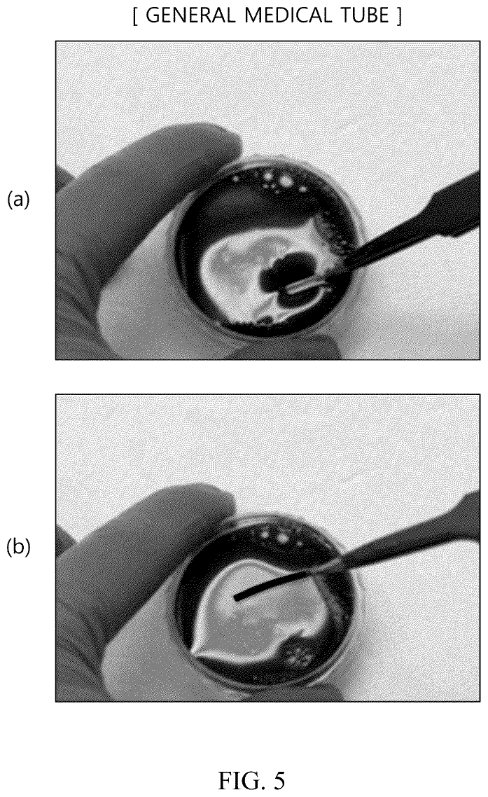

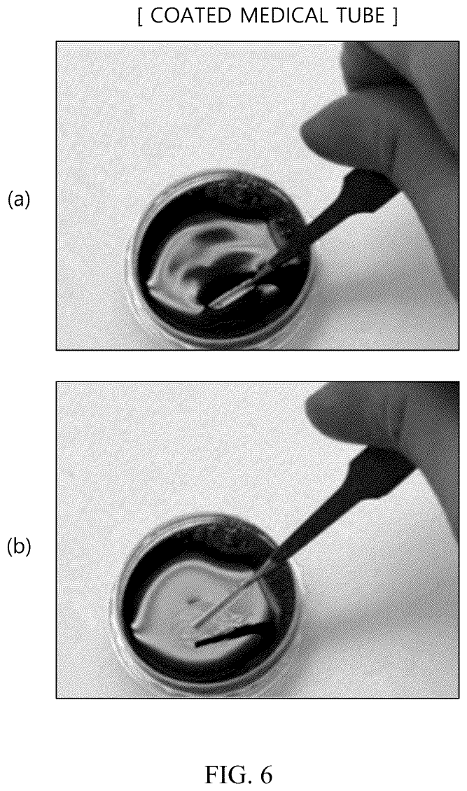

To evaluate the foreign substance adhesion prevention effect, a blood immersion test was conducted on the coated medical tube prepared in Example 2 and the uncoated medical tube of Comparative Example 2. The results are shown in FIGS. 5 and 6, respectively.

Specifically, FIGS. 5 and 6 show a sequence of photographs illustrating the process of immersing and removing medical tubes from a blood-containing container. FIGS. 5(a) and 6(a) each depict the surface of the medical tube immersed in blood, while FIGS. 5(b) and 6(b) show the surface of the medical tube after removal from the blood.

As shown in FIG. 5(b), in the case of the uncoated medical tube, blood remained attached to the surface, confirming contamination of the medical tube. In contrast, as seen in FIG. 6(b), the medical tube coated with the coating composition of the present invention did not retain blood on its surface after removal, demonstrating that the tube remained uncontaminated.

Therefore, when the coating composition of the present invention is applied to stents, catheters, and other medical tubing, it provides a foreign substance adhesion prevention effect and a contamination prevention effect against substances such as blood. Consequently, this is expected to reduce the risk of infection and inflammation in patients.

What is claimed is:

1. A coating composition for an implantable medical device comprising:
a stock solution and a diluted solution,
wherein the stock solution includes
an aliphatic hydrocarbon solvent;
a siloxane compound;
an alcohol;
one or more amino silanes; and
a silane-based oligomer.

2. The coating composition for an implantable medical device of claim 1, wherein the diluted solution includes an organosilicon compound.

3. The coating composition for an implantable medical device f claim 1, wherein a weight mixing ratio of the stock solution and the diluted solution is 1:45 to 55.

4. The coating composition for an implantable medical device of claim 1, wherein the implantable medical device is selected from the group consisting of a stent, a catheter, a subcutaneous implant, a chemical sensor, a lead, a pacemaker, a vascular graft, a dressing, an implantable pulse generator, and a cardiac defibrillator.

5. The coating composition for an implantable medical device of claim 4, wherein the stent is selected from the group consisting of an esophageal stent, a vascular stent, a biliary stent, a gallbladder stent, a pancreatic stent, a uterine stent, a urethral stent, a ureteral stent, a lacrimal stent, an Eustachian tube stent, a fallopian tube stent, and a bronchial stent.

6. The coating composition for an implantable medical device of claim 4, wherein the catheter is selected from the group consisting of a drainage catheter, an infusion catheter, a parenteral feeding catheter, and a urological catheter.

7. An implantable medical device coated with the coating composition of claim 1.

8. An implantable medical device coated with the coating composition of claim 2.

9. An implantable medical device coated with the coating composition of claim 3.

10. An implantable medical device coated with the coating composition of claim 4.

11. The implantable medical device of claim 7, wherein the implantable medical device is further coated with a silane-based lubricant.

12. A method of coating an implantable medical device, comprising administering the coating composition according to claim 1 to a surface of the implantable medical device to coat the surface.
